(19) United States
(12) Patent Application Publication
(10) Pub. No.: US 2015/0354000 A1
(43) Pub. Date: Dec. 10, 2015

(54) METHOD OF ANALYSIS OF COMPOSITION OF NUCLEIC ACID MIXTURES

(71) Applicant: MAX-PLANCK-GESELLSCHAFT ZUR FÖRDERUNG DER WISSENSCHAFTEN E.V., Munich (DE)

(72) Inventors: Tatiana BORODINA, Berlin (DE); Aleksey SOLDATOV, Berlin (DE); Hans LEHRACH, Berlin (DE)

(21) Appl. No.: 14/655,948

(22) PCT Filed: Dec. 31, 2013
(86) PCT No.: PCT/EP2013/078177

§ 371 Date: Jun. 26, 2015

(30) Foreign Application Priority Data
Dec. 28, 2012 (EP) 12199784.5

(51) Int. Cl. C12Q 1/68; C12N 15/10

(52) U.S. Cl. CPC C12Q 1/6869; C12N 15/1065

(57) ABSTRACT

When sequencing is used for the analysis of composition of nucleic acid mixtures with a large dynamic range of concentrations of individual components, the reliability of results significantly differs for abundant and rare components. The present invention relates to methods for analysis of concentrations of components of nucleic acid mixtures by sequencing, wherein relative abundances of at least two components for which concentrations should be measured is changed before sequencing in a reproducible way using locus-specific oligonucleotides.

### FIELD OF THE INVENTION

When sequencing is used for the analysis of composition of nucleic acid mixtures with a large dynamic range of concentrations of individual components, the reliability of results differs significantly for abundant and rare components. This is a common problem for studying of transcriptomes and for analysis of biodiversity by sequencing of environmental and clinical samples. We suggest a method of analysis which allows adjusting the reliability of results individually for each component of the nucleic acid mixture in a highly reproducible manner: Controllable Oligonucleotide-Based Ratio Adjustment (COBRA). The method is based on using locus-specific oligonucleotides to change the relative abundance of individual components of nucleic acid mixture before sequencing.

The method is especially useful for routine analysis of biodiversity and routine expression profiling, like for clinical studies.

### BACKGROUND OF THE INVENTION

RNA-Seq (RNA Sequencing) is a hypothesis-free approach for studying of transcriptome by sequencing of millions of cDNA fragments. The abundance of cDNA fragments matches the abundance of the corresponding transcript. The obtained sequencing results give a possibility to retrieve information about abundance and structure of transcripts.

RNA-Seq is complicated by two problems:
1. Gene expression levels have a huge dynamic range (about 5 orders of magnitude). So, in order to characterize low-expressed genes it is necessary to over-sequence highly expressed ones. The more sequencing reads correspond to a particular transcript, the more reliably its expression level is determined.
2. It is difficult to estimate accurately the expression level of similar transcripts. Similarity of transcripts is a common phenomenon:
   all genes have two (or more in case of polyploid organisms) homologous copies (alleles);
   repetitive genomic regions give rise to similar transcripts;
   individual genes may produce several similar transcripts (splice variants) due to presence of alternative donor- and acceptor-splicing sites.

Only a portion of reads mapped to the similar transcripts may be used for characterization of expression levels of individual homologues: namely those reads which overlap sites, different between the homologues. Other reads may be used only for characterization of cumulative expression level.

Usually only a part of RNA-Seq library is sequenced. Concentration of abundant transcripts is determined with excessive reliability, but concentration of rare transcripts only with insufficient reliability. Sequencing of the rest of the library would improve the reliability of measurement of concentration of rare transcripts. But only a small part of the additional sequencing reads would correspond to rare transcripts, most of the additional sequencing reads would correspond to abundant transcripts.

It would be more attractive to reduce the number of sequencing reads corresponding to abundant transcripts (which are analyzed with redundant reliability). In this case more reads would correspond to rare transcripts and reliability of analysis of rare transcripts would increase.

### DESCRIPTION OF THE INVENTION

COBRA-Approach

In this invention we suggest to change the way how massively parallel sequencing is used for the analysis of mixtures containing different nucleic acids, in particular for determination of concentrations of individual components.

Currently, a sequencing library is prepared from the mixture under analysis by such a way, that the relative abundances of the individual components in the library match as close as possible to the abundance of the corresponding components in the mixture under analysis. Thus, when sequencing reveals abundances of the components of the sequencing library it also determines the abundance of the components in the mixture under analysis. The problem is that the reliability of results significantly differs for abundant and rare components.

We suggest preparing sequencing libraries, in which abundances of individual components are selectively and controllably modified (FIG. 1). For selective and controllable modification of abundance we suggest to use locus-specific oligonucleotides (Controllable Oligonucleotide-Based Ratio Adjustment COBRA).

The idea is to controllably and reproducibly modify the abundances of some components of the mixture before sequencing: to decrease the abundances of those components, which are analyzed with excessive reliability and/or to increase the abundances of those components, which are analyzed with insufficient reliability. As a result the desirable accuracy of concentration measurement (for all analyzed components) would be achieved with less sequencing reads if compare with sequencing without preliminary abundance modification.

Locus-specific oligonucleotides allow to affect independently individual components of nucleic acid mixture. As soon as we can address individual components we can apply a number of molecular biology techniques to vary effectiveness of converting of molecules of the analyzed mixture into the molecules of sequencing library.

In this application we describe three methods for reproducible and predictable regulation of abundance of sequencing library molecules correspondent to different components of nucleic acid mixture:
1. Selection of different number of detectable loci for different components of nucleic acid mixture (see FIG. 2).
2. Combining of loci in several groups according to a desirable “abundance change factor” and using of different library-preparation protocols for different groups (see FIG. 3, Examples 3-7).
3. Using a mixture of “functional”/“blocked” oligonucleotides to adjust “abundance change factor” individually for each detectable locus (see FIG. 4, Examples 8-12).

It is quite possible that there are other methodological solutions for COBRA-approach. But even these three approaches and their combinations provide a variety of protocols for preparation of COBRA sequencing libraries.

The present invention refers in particular to a method for analysis of concentrations of components of nucleic acid mixtures by sequencing, wherein relative abundances of at least two components for which concentrations should be measured is changed before sequencing in a reproducible way using locus-specific oligonucleotides and wherein said change of abundances comprises the following steps:
i) selection of at least two nucleic acid components of the original mixture for which concentrations should be measured and relative abundances should be changed and designing locus-specific oligonucleotides for said at least two nucleic acid components;
ii) creation from original nucleic acid mixture a subsequent nucleic acid mixture wherein relative abundances of components corresponding to the components selected on step i) are changed in reproducible manner using said locus-specific oligonucleotides designed on step i).

Within the methods of the present invention the analysis of concentrations of components of nucleic acid mixtures with changed abundance by sequencing takes place subsequently to step ii). Thus, the present invention refers to a method for analysis of concentrations of components of nucleic acid mixtures by sequencing, wherein relative abundances of at least two components for which concentrations should be measured is changed before sequencing in a reproducible way using locus-specific oligonucleotides and wherein said method comprises the following steps:
i) selection of at least two nucleic acid components of the original mixture for which concentrations should be measured and relative abundances should be changed and designing locus-specific oligonucleotides for said at least two nucleic acid components;
ii) creation from original nucleic acid mixture a subsequent nucleic acid mixture wherein relative abundances of components corresponding to the components selected on step i) are changed in reproducible manner using said locus-specific oligonucleotides designed on step i)
iii) analysis of concentrations of components of nucleic acid mixtures with changed abundance by sequencing.

Within the inventive method it is preferred that the relative abundances of components corresponding to the components selected on step i) are changed on step ii) by
a) using differing number of locus-specific oligonucleotide sets for said components,
and/or
b) using for these components differing reaction conditions,
and/or
c) using for these components mixtures of functional and blocked locus-specific oligonucleotides with differing ratio of said “functional to blocked” locus-specific oligonucleotides,
and/or
d) by using for these components locus-specific oligonucleotides with differing concentrations or with differing efficiency of hybridization.

Preferred are methods according to the present invention, wherein relative abundances of components selected on step i) are changed in such a way, that the dynamic range of concentrations of components under analysis in the subsequent nucleic acid mixture is lower than the dynamic range of concentrations of components under analysis in the original mixture containing nucleic acids or in a way which decreases the abundance of components for which concentration without change of abundances is measured with excessive accuracy and/or increases the abundance of components for which it is desirable to increase the accuracy of concentration measurement if compared with measurement of concentration without change of abundances.

The present invention refers further to a method of analysis of concentrations of nucleic acid components in mixtures containing nucleic acids, comprising the following steps:
i) providing an original mixture containing nucleic acids;
ii) selection of at least one nucleic acid component of the original mixture for which the abundance should be changed in predefined manner;
iii) creation from original mixture containing nucleic acids a subsequent nucleic acid mixture wherein abundances of components corresponding to the components selected on step ii) are changed in predefined manner using locus-specific oligonucleotides for said components;
iv) analysis of concentrations of at least two components in the subsequent nucleic acid mixture for which relative abundances were changed in predefined manner compared with relative abundances of corresponding components in the original mixture containing nucleic acids.

An alternative formulation for this method the present invention refers to is:
Method for analysis of concentrations of nucleic acid components in mixtures containing nucleic acids, comprising the following steps:
i) providing an original mixture containing nucleic acids;
ii) choosing at least two nucleic acid components of the original mixture for which the relative abundance should be changed in predefined manner and designing component-specific oligonucleotides specifically for said at least two nucleic acid components;
iii) creation from original mixture containing nucleic acids a subsequent nucleic acid mixture wherein relative abundances of components corresponding to the components selected on step ii) are changed in reproducible manner using designed component-specific oligonucleotides for said components;
iv) analysis of concentrations of components of subsequent nucleic acid mixture wherein the concentrations are measured for at least two components of those for which relative abundances were changed and wherein concentrations measured for the at least two components are representative for the concentration of corresponding nucleic acids in the original mixture.

To determine concentrations of components in the original nucleic acid (NA) mixture their concentrations in the sequencing library should be multiplied on corresponding abundance change factors. Thus it is possible to compare not only experiments of the same series between each other, but also the experiments performed by different people using different COBRA-based protocols.

Because the relative abundances of the at least two components for which concentrations should be measured is changed in a reproducible and preferably also predictable way it is possible to calculated the concentration of the component in the original mixture using division by correspondent abundant change factors. Preferred are methods according to the invention, wherein relative concentrations of components under analysis in the original nucleic acid mixture are calculated by dividing results obtained after changing of abundances by correspondent abundant change factors.

Locus-specific oligonucleotides allow not only to affect individual components of a mixture of nucleic acids but also to select for sequencing certain parts of these components to avoid difficult-for-analysis regions. For expression profiling locus-specific oligonucleotides give a possibility to select for sequencing only non-repetitive regions of genes. For analysis of biodiversity it is preferred to exclude from the sequencing library evolutionary conserved regions.

Using of locus-specific oligonucleotides allows to combine the selectivity of microarrays with the accuracy and sensitivity of massive parallel sequencing. As in microarray technologies, COBRA procedure requires hundreds and thousands of locus-specific oligonucleotides. That is why COBRA procedure may be not relevant for preparation of single libraries. But for a massive screening or for routine analyses, large set of locus-specific oligonucleotides is not a big inconvenience, because such set should be prepared only once.

Besides, for a lot of applications, the COBRA oligonucleotide set is determined mainly by the type of tissue under analysis, because particular a type of tissue defines which genes are over-expressed and consequently over-sequenced. In clinical analyses only a few types of human tissues are easily available (such as blood, saliva, buccal cells, sperm). For each of these tissues, appropriate locus-specific COBRA oligonucleotides may be designed.

Practical Implementation

Although we propose to use, for analysis of nucleic acid mixtures, a new type of libraries (with altered abundances of individual components), it does not mean that new molecular methods are needed. Already known and proven approaches can be adapted for COBRA. Two issues are required for adaptation:
Locus-specific oligonucleotides should be used for preparation of the library (to have a possibility to affect individual components of the mixture); and
it should be selected a procedure for reproducible modification of the abundances of components.

Locus-specific oligonucleotides are widely used in biomedicine. They allow specifically targeting components with definite known nucleotide sequences in complex mixtures of nucleic acids. Specificity of targeting is based on specificity of hybridization of nucleic acids: the most stable hybrid is formed with perfectly matched sequences. Locus-specific oligonucleotides provide specificity of many types of molecular biology reactions:
amplification, for example PCR, BRCA (Branched Rolling-Circle Amplification), LCR (Ligase Chain Reaction);
detection, for example gap-filling extension-ligation, DANSR (digital analysis of selected regions), Northern blots, Southern blots, microarray hybridization for SNP detection or expression profiling;
target-enrichment strategies for next-generation sequencing

All these methods are associated with some background because of unspecific hybridization. Unspecific hybridization may appear because of repetitive regions of the genome. Besides, some completely unique sequences may interact too strong with not perfectly matched sequences. But for all mentioned procedures and for most non-repetitive regions a person skilled in the art is capable to select locus-specific oligonucleotides which provide acceptable background level. In case of analyzing of results by sequencing, significant part of non-specific products may be eliminated on analysis stage, for example, because extension reaction results in wrong nucleotide sequence or incorrect primer combination appeared as a result of ligation.

The term “locus-specific oligonucleotides” or “site-specific oligonucleotides” as used herein refers to a short, chemically synthesized nucleic acid complementary to the sequence of a site in the component of the nucleic acid mixture. The locus-specific oligonucleotides hybridize in a sequence-specific manner to a specified locus, portion or region of a selected component of the nucleic acid mixture. Therefore the locus-specific oligonucleotides can be used to determine the locus, region or fragment of the selected component of the nucleic acid mixture. The locus, region or fragment is determined to be targeted by a subsequent enzymatic reaction such as amplification or sequencing. Locus specific oligonucleotides may be for example: primer as a starting point for DNA synthesis (eg during PCR), probes or oligonucleotides for hybridization or ligation reactions.

If a library preparation method is already using locus-specific oligonucleotides, it is possible to use those oligonucleotides for regulation of the abundances of correspondent sequencing library molecules. For example, Illumina TruSeq™ Targeted RNA Expression Kits is based on extension/ligation of locus-specific oligonucleotides on cDNA. These oligonucleotides can be used as an instrument for abundance regulation.

If there are no locus-specific oligonucleotides in the protocol, it is possible to introduce them at some stage. Classic protocol for preparing RNA-Seq libraries does not involve any locus-specific oligonucleotides. But they may be included in the protocol, for example, in the following way:
for positive selection of RNA molecules by hybridization before library preparation;
for negative selection of RNA molecules by hybridization before library preparation (cf. Example 12);
as primers for the first strand synthesis (cf. Example 8);
for positive selection of ready-to sequencing library molecules by hybridization before sequencing (cf. Examples 7, 11).

The following paragraphs describe the second issue necessary for implementation of COBRA-libraries, namely procedures for reproducible and predictable modification of the abundances. Three approaches with easily predictable abundance change factors are described in detail: (i) using different number of loci per transcript; (ii) using of different library-preparation protocols for different groups of loci; (iii) using a mixture of “functional” and “blocked” locus-specific oligonucleotides. Besides, approaches are outlined for which it is difficult to predict in advance the abundance change factors, but which can provide reproducible change of abundance.

Using a method according to the invention a subsequent nucleic acid mixture is created which is preferably selected from the group comprising or consisting of: sequencing library, set of ligated locus-specific oligonucleotides, set of locus-specific oligonucleotides extended in a template-dependent reaction, set of fluorescently labeled molecules, nucleic acids molecules selected with the help of hybridization with locus-specific oligonucleotides.

Number of Detectable Loci per Transcript

If not one but several detectable loci or sites (preferably, located in a way that they do not compete with each other during library preparation) are selected for a certain component of the nucleic acid mixture, the number of sequencing reads matching this component would increase proportionately. This will increase the reliability of concentration measurement of the component.

Selection of a different number of detectable loci for regulation of abundance of correspondent molecules in sequencing library has certain advantages and disadvantages.

Advantages:
the method is fully compatible with other COBRA-approaches and library preparation protocols;
the method allows easily adjustment of the abundance in a small range (up to about 10);
in contrast to most other COBRA-approaches, this method does not reduce but increases the abundance.

Disadvantages:
the number of loci may be limited for some nucleic acid components (especially for components having homologues, where loci should be located in regions being different between these homologues);
regulation is stepwise;
the method is not suitable for suppression, only for increasing the abundance;
the synthesis of new locus-specific oligonucleotides is required to change the level of regulation.

Combining Loci in “Change of Abundance” Groups

If it is not necessary to provide a precise value of abundance change factor for each selected component, loci with similar required adjustment levels may be combined in groups. Then the COBRA-library may be planned as following:

a) select the desired abundance change factor for each locus;

b) combine loci with similar abundance change factors into groups and choose a common factor for each group;

c) select for each group of loci a library preparation protocol with the required abundance change factors value.

Groupwise regulation of the relative abundances allows reducing the dynamic range of concentrations. One can for example combine transcripts in three groups: “without suppression”, “10× suppression” and “100× suppression,” according to their expression level, than the dynamic range is reduced from five to three orders of magnitude (FIG. 3).

Locus-specific oligonucleotides corresponding to different adjustment levels (and participating in different protocols) should be somehow grouped. This can be done in two ways:
by spatial isolation: the locus-specific oligonucleotides may be assembled in separate tubes according to adjustment levels; or
by labeling: locus-specific oligonucleotides from different groups may be combined together, if group-specific markers are introduced into oligonucleotides.

Spatial isolation of locus-specific oligonucleotides enables performing of spatially isolated reactions. Library preparation reactions correspondent to different adjustment level groups may be completely independent from each other or differing only by a certain stage. Independent preparation of libraries for loci with different adjustment levels gives a full freedom in choosing the protocol (different principles, different enzymes), but requires more labor and can lead to unstable results of comparison of expression levels of genes from different adjustment level groups. Minimizing the number of differing stages decreases labor costs and makes the comparison of abundances of different components more reproducible. A spatially separated stage can be introduced at any point of the library preparation protocol:
in the beginning—for example, separate aliquots of the original mixture for different groups (Examples 3, 4);
in the middle part of the preparation protocol—for example, different conditions of pre-amplification of library molecules belonging to different adjustment level groups (see Example 6);
at the end—for example, classical RNA-Seq reaction, followed by separate hybridization-based selection of library molecules with oligonucleotides belonging to different adjustment level groups (see Example 7).

The reaction conditions would be as similar as possible, if locus-specific oligonucleotides for different groups are added subsequently to the same reaction (see Examples 5 and 6).

Markers of abundance level correction introduced in the locus-specific oligonucleotides allow to minimize differences in the reaction conditions and even to synthesize a sequencing library for all groups together. There are a variety of experimental realizations of using marker regions for abundance level correction, which vary from primitive like “divide the mixture into fractions by hybridization with a marker region and then take the appropriate part of the volume of each fraction”, to sophisticated methods like marker-specific PCR with different number of cycles for different markers (see the next paragraph).

Groupwise abundance level correction has certain advantages and disadvantages. Advantages are:
convenient approach for wide range regulation of abundance;
modified locus-specific oligonucleotides are not required (if compare with using functional and blocked locus-specific oligonucleotides);
any degree of suppression/enhancement can be reached and accurately reproduced. For example: using serial dilutions it is possible to take accurately 1/10000 of the reaction mixture; 10 cycles of PCR amplification give a quite accurate enhancement in 1000 times;
abundance change factor for the group as a whole can be easily changed.

Disadvantages are:
stepwise regulation and the number of steps is limited;
reactions with different groups of locus-specific oligonucleotides are performed separately which reduces the reliability of the method and makes it dependent on the precision/accuracy of the separation of the mixture;
transferring a locus from one group to another requires regrouping of locus-specific oligonucleotides.

One aspect of the present invention is that the relative abundances of components corresponding to the components selected on step i) are changed on step ii) by using for these components differing reaction conditions. Thereby it is preferred that said differences in reaction conditions are selected from the group consisting of or comprising: different amounts of original mixture containing nucleic acids used in reactions; different number of cycles in cyclic amplification reactions; different reaction times in linear amplification reactions. Implementation of different reaction conditions may comprise grouping of several components selected in step i) according to similar abundance change factor.

Functional and Blocked Locus-Specific Oligonucleotides

FIG. 4 shows how a mixture of functional and blocked locus-specific oligonucleotides allows adjusting abundance individually and independently for each locus of a nucleic acid component. Functional and blocked locus-specific oligonucleotides may be designed using the following principle: “blocked” oligonucleotides should compete with “functional” oligonucleotides in the same reaction, but blocked oligonucleotides either block the reaction, or the reaction products obtained from blocked oligonucleotides can be separated from the reaction products obtained from functional oligonucleotides.

In fact, any oligonucleotide, which competes with the locus-specific oligonucleotides suppress the reaction. But when blocked and functional locus-specific oligonucleotides have the same nucleotide sequences, the degree of suppression is easily predictable, determined only by the ratio of concentrations of functional to blocked locus-specific oligonucleotides and do not depends on the reaction conditions (temperature, time, buffer, etc.). Thus it is preferred that the functional and blocked locus-specific oligonucleotides specific for a certain locus or site of a component have an identical sequence.

The ratio of functional to blocked oligonucleotides can be selected independently for each locus of the selected nucleic acid component. As a result, the efficiency of conversion of original molecules into molecules of the library can be tuned independently for each locus.

Different blocking approaches may be used for locus-specific oligonucleotide-dependent reactions:
primer extension: blocking of 3′ end of primers (e.g. 3′ amino-modified primer);
ligation: blocking of 3′ end of upstream primers (e.g. 3′ amino-modified primer); blocking of 5′ end of downstream primers (e.g. 5′ dephosphorylated primer);
hybridization-based selection: using oligonucleotides without a complementary region (e.g. region for hybridization or for PCR-amplification);
affinity selection: using oligonucleotides without affinity region (e.g. biotinylated/non-biotinylated locus-specific oligonucleotides).

According to the invention it is preferred that the relative abundances of components corresponding to the components selected on step i) are changed on step ii) using for these components mixtures of functional and blocked locus-specific oligonucleotides with differing ratio of said “functional to blocked” locus-specific oligonucleotides. Thereby it is further preferred that the functional locus specific oligonucleotides can while the blocked locus specific oligonucleotides cannot be elongated in reaction of primer extension, or reaction of first-strand synthesis, or reaction of second-strand synthesis, or in PCR, or in gap-filling reaction because they have 3′ end modification.

One further aspect of the present invention relates to methods wherein the functional oligonucleotides can while blocked oligonucleotides cannot participate in ligation steps of ligation detection reaction, or in gap-filling reaction, or in LCR, or in DANSR because they have 3′ or 5′ end modifications.

One further aspect of the present invention relates to methods wherein functional and/or blocked locus-specific oligonucleotides have markers. These markers allow separating of subsequent molecules containing the functional locus-specific oligonucleotides or their marker from subsequent molecules containing the blocked locus-specific oligonucleotides or their markers. Such subsequent molecules can for example be hybrids of functional locus-specific oligonucleotides with target nucleic acid components or products of reaction involving functional oligonucleotides and respectively hybrids of blocked locus-specific oligonucleotides with target nucleic acid components or products of reaction involving blocked locus-specific oligonucleotides.

Functional and blocked locus-specific oligonucleotides producing separable reaction products, allow to work both with suppressed sequencing library (after removal of the sequencing library molecules synthesized by using the “blocked” primers), and with non-suppressed sequencing library (without separation of the sequencing library molecules synthesized by using the “blocked” primers). Besides, enzymatic reactions are provided with a high concentration of substrate at all stages of library preparation (some enzymes do not work well with the substrate at low concentrations).

Different approaches allow to separate the reaction products obtained from functional and blocked locus-specific oligonucleotides, for example:
when using biotinylated functional or blocked primers the resulting or corresponding products can be attached to streptavidin-coated surfaces and further separated from the mixture;
when blocked locus-specific oligonucleotides contain deoxyuridine, the corresponding products can be destroyed by UDGase (uracil-DNA glycosylase). Similarly, when methylated functional and unmethylated blocked locus-specific oligonucleotides are used only unmethylated products could be digested by methylation-sensitive restriction enzymes;
functional locus-specific oligonucleotides with conservative terminal regions (for further amplification with common primers; standard or commonly used sequences for sequencing primers such as M13, T7, poly A or polyT) and blocked locus-specific oligonucleotides not containing this region can be used.

Therefore within the methods of the present invention it is preferred that functional and/or blocked locus-specific oligonucleotides have markers selected from the group comprising or consisting of:
presence in oligonucleotide of dUTP for subsequent specific destruction;
presence in oligonucleotide of thio-modified bonds for subsequent specific destruction;
presence in oligonucleotide of biotin for subsequent specific affinity selection;
presence in oligonucleotide of 5-bromo-2′-deoxyuridine (BrdU) for subsequent specific affinity selection;
presence in oligonucleotides of sequence specific for subsequent amplification or hybridization-based selection.

Advantages of COBRA methods based on using a mixture of functional and blocked locus-specific oligonucleotides are:
independent regulation of suppression level for each individual locus;
change of the regulation level does not require synthesis of new locus-specific oligonucleotides;
library preparation reactions are performed in one mixture using the same conditions;

Disadvantages are:
except for the usual set of functional locus-specific oligonucleotides, at least one additional blocked locus-specific oligonucleotide is required for each locus;
change of the regulation level requires redesign of the mixture of locus-specific oligonucleotides.

“Abundance change factor” is introduced to characterize the amount of change of relative abundance of an individual component of the nucleic acid mixture. It is calculated by dividing of relative abundance of this component after changing of abundances on relative abundance of the component before changing of abundances. Thus, if 80% of the copies of one particular component in the nucleic acid mixture are blocked because the ratio of functional to blocked locus-specific oligonucleotides is 1:4 the abundance change factor for this component is 0.2. Abundance change factor for the component is 1 if the relative abundance for this component did not change.

Other Approaches for Regulation of Abundance Using Locus-Specific Oligonucleotides

In the three approaches described above the abundance change factor is known in advance. For example when two detectable loci are selected instead of one for a certain component of the nucleic acid mixture, the abundance of this component in a sequencing library increases two times and the abundance change factor is 2. For a 1:1 mixture of functional to blocked locus-specific oligonucleotides a two times decrease of the abundance of the corresponding component in the sequencing library takes place. This feature (predictability of abundance change factor) is convenient, but not obligatory for preparation of libraries with modified abundances of components.

It is possible to use such techniques for changing of abundance of components, for which the abundance change factor is difficult to predict theoretically but can be revealed experimentally. The main thing is that abundance change factors remain the same in different experiments. If necessary, values of abundance change factors may be determined in the control experiment. Below we describe some examples of such techniques.

Functional and Blocked Locus-Specific Oligonucleotides with Differing Nucleotide Sequences.

When the nucleotide sequences of functional and blocked locus-specific oligonucleotides are identical, the abundance change factor depends only on the ratio of concentrations of functional and blocked locus-specific oligonucleotides and remains the same under any experimental conditions. Blocked locus-specific oligonucleotides with non-identical length or with non-identical nucleotide sequence (if compare to “functional”) still would suppress the conversion of components of analyzed mixture into the library molecules, but suppression rate would somehow depend on reaction conditions (temperature, buffer, etc.). Nevertheless, providing standard conditions it may be possible to preserve the same abundance change factors in different experiments. Thus, functional and blocked locus-specific oligonucleotides with different sequences can also be used for the preparation of COBRA-libraries.

Locus-Specific Oligonucleotides with Impaired Hybridization Properties.

It is possible to change the nucleotide sequence of locus-specific oligonucleotides (nucleotide substitutions, change the length), in order to weaken binding of oligonucleotides to the template and thus to suppress the conversion of correspondent components of analyzed mixture into the library molecules. Suppression level is hardly predictable, but it may be determined in a control experiment.

Change of Concentration of Locus-Specific Oligonucleotides

Influence of concentration of locus-specific oligonucleotides on the efficiency of conversion of components of the analyzed mixture into the library molecules is nonlinear and difficult to predict. But from general considerations it is clear that decreasing the concentration would at some point lead to the suppression of the conversion of components of analyzed mixture into the library molecules. Suppression level can be set up in control experiments.

It is possible to use a combination of abundance change methods. FIG. 10 shows an example of combination of “abundance correction groups” and “functional/blocked locus-specific oligonucleotide” approaches. Let's assume that there is a kit for preparation of COBRA RNA-Seq libraries. Locus-specific oligonucleotides in the kit are divided into two sets: “abundant” and “rare”. When using all locus-specific oligonucleotides in the common reaction, the number of clones per locus is about 10 times higher for the abundant group, if compared with the rare group. This is useful, because otherwise for the limited amount of starting material low fidelity results would be obtained both for the abundant and for the rare loci. However, when there is an excess of starting material and the results for the rare group are quite reliable, it is not practical to maintain a ten-fold excess for the abundant group. Then it makes sense to use the abundant set of oligonucleotides for the synthesis of libraries with less starting material to level off the representation.

Therefore the present invention refers to a kit, suitable for analysis of concentrations of nucleic acid according to any one of claims 1-13, which produce from original mixture containing nucleic acids some subsequent nucleic acid mixture, wherein abundance of definite set of components is decreased in reproducible manner using functional and blocked locus-specific oligonucleotide sets.

Discussion

Sequencing is one of the most powerful methods of analysis of nucleic acid mixtures. The method allows to identify composition of nucleic acid mixtures and to determine concentrations of individual components. In this case the sequencer is used not for revealing of the unknown nucleotide sequences, but for recognizing of the known molecules. Analysis of concentrations of components of nucleic acid mixtures by sequencing is widely used for studies of biodiversity and expression profiling in medicine, veterinary, agriculture, and ecological studies.

Expression profiling is used for analysis of mixtures of RNA molecules: which molecules are present in the mixture and in what proportion. Sequencers cannot read RNA molecules directly. First, RNA molecules have to be converted into sequencing library molecules. Depending on the method, different parts of RNA molecules are converted into sequencing library molecules (DNA): random fragments of RNA molecules (RNA-Seq method), terminal regions of RNA molecules (5′- or 3′-terminal regions), or specifically selected internal fragments of RNA molecules (e.g. Illumina TruSeq™ Targeted RNA Expression Kits). Sequencing libraries may contain a very large number of molecules. The entire library or some portion of the library is sequenced. Usually not the full-length library molecule but just a part of it is sequenced (depending on the type and operation mode of a sequencer).

Certain efforts are required to get from a set of sequencing reads information about composition of the mixture and concentration of its components. Each read should be associated with the corresponding transcript. More reads are associated with highly expressed transcripts, less reads—with weakly expressed. Frequency of read occurrence is directly proportional to the abundances of corresponding transcripts.

Sequencing provides relative abundances of transcripts (usually referred to as the number of a certain type of transcripts per million of RNA molecules). Additional work is required to determine the absolute number of transcripts per cell. Analysis of the mixture by sequencing is very sensitive and specific. Even only one rare molecule in the initial mixture has a chance to be sequenced and accurate sequencing would leave no doubt that the transcript is exactly identified. Very similar isoforms can be distinguished, by sequencing of the differing regions.

In practice, there may be problems both with the identification of molecules and with calculation of their abundances. For accurate identification of molecules it is necessary to know the nucleotide sequences of possible transcripts. Inaccurate description of the transcriptome in the database will cause problems with identification of sequencing reads. Reads corresponding to the repetitive regions cannot be unambiguously ascribed to certain transcripts. Recognition of transcripts from organisms with large genomes requires analysis of large volumes of data, use of powerful computers and complex algorithms.

The main problem in determining of abundances is rare transcripts. In principle, the concentration analysis by sequencing is a scalable method. The greater the total number of reads, the more accurately rare transcripts will be analyzed. The problem is that the bulk of additional reads would correspond to common transcripts for which the abundances are already determined with sufficient accuracy.

Another problem is that in the course of RNA isolation and library preparation the abundances of transcripts are distorted. This may be due to the different efficiency of isolation of long and short RNA molecules, different conversion efficiency of RNA molecules into library molecules (5′- regions are less effectively converted into cDNA, than 3′- regions) or with different efficiencies of amplification during library preparation (amplification is dependent on GC- composition, presence of palindromes, etc.). For proper evaluation of abundances it is also necessary to consider that longer transcripts give more library molecules than shorter, unique sites results in more recognizable library molecules than areas with repeats, areas of RNA with secondary structure results in less library molecules than areas without it and so on. Not all of these factors are taken into account in practice, and abundances of transcripts are systematically over- or underrepresented. This is not a problem, since in most cases researchers are interested not in the absolute values of abundances, but in how changes in transcription level correlate with various biomedical effects. For example, how gene expression levels change in tumor tissue compared to healthy tissue, or how gene expression levels change in an ill patient compared to healthy persons. Accordingly, not absolute but relative abundances are normally of interest: the ratios of expression levels in the sample to the expression levels in the control.

The emergence of new generations of sequencing technologies significantly reduced sequencing price per nucleotide, but did not change the fact that the bulk of the funds during massive screenings is still spent particularly on sequencing. Introduction of COBRA-approach may improve the sequencing efficiency in routine clinical and environmental analyses and in research studies.

During routine clinical and environmental analyses part of the sequencing data is useless, such as:
redundant sequences of overrepresented components,
data from difficult-to-interpret regions (repeats, low-complexity regions),
sequences of regions which are of no interest to the investigator.

COBRA approaches with a positive selection (wherein sequencing library contains only components corresponding to locus-specific oligonucleotides, because all other nucleic acid components of original mixture are lost) allow to solve these problems and to provide the following advantages:
decrease the relative abundances of overrepresented components and consequently increase the relative abundance of underrepresented components;
select for sequencing only informative regions;
select for sequencing only a defined list of genes.

Besides, positive selection allows to get rid of ribosomal RNA, which is especially convenient for the analysis of bacterial transcription, where polyA⁺ selection cannot be applied.

As a result, useless sequencing results will be eliminated and it would be possible to achieve the same accuracy of concentration measurements with a smaller total number of sequencing reads.

Therefore within the present invention one preferred aspect are methods wherein subsequent nucleic acid mixture is created by positive selection with locus-specific oligonucleotides and contains only components corresponding to locus-specific oligonucleotides while all other nucleic acid components of original mixture are removed.

An essential requirement for research studies is the hypothesis-free nature of the analysis so that information about all components of the mixture should be obtained. The sources of useless sequencing results in research studies are:
redundant sequences of overrepresented components,
data from difficult-to-interpret regions (repeats, low-complexity regions).

Positive selection can't be applied for research studies, where it is not known in advance which component of the mixture is important. But it is possible to apply negative COBRA selection, where locus-specific oligonucleotides are used to reduce the number of unwanted nucleic acid components and sequencing library preserves all components which have no corresponding locus-specific oligonucleotides.

Negative COBRA selection has the following advantages:
it is a hypothesis-free approach;
if the negative selection is applied for the change of composition of starting material, the procedure is easily compatible with any sequencing library preparation protocol;
the procedure can be combined with the removal of ribosomal RNA.

Therefore within the present invention another preferred aspect are methods wherein subsequent nucleic acid mixture is created by negative selection with locus-specific oligonucleotides and in the subsequent nucleic acid mixture relative abundances are changed only for components corresponding to locus-specific oligonucleotides.

The goal of changing abundances within the inventive methods is not to bring all components to the same concentrations. The idea of the COBRA approach is to provide a possibility to the researcher to choose the reliability of abundance measurement depending on the experimental goal and on properties of the biological system under study. Different nucleic acid components may:
be of different interest for a researcher—for example, expression levels of some genes are important for making a decision in clinical analysis and it is desirable to know them with high accuracy, whereas some others may serve only as general controls—for those high accuracy is not needed. Some genes may be excluded from the analysis completely.
have different distributions in biological system under study. For example, if it is known that the concentration of the first transcript varies within 10% in different biological samples, and the concentration of the second transcript may differ in two times, it makes no sense to measure the concentration of the second transcript with the same accuracy as for the first. The concentration of the first transcript should be measured more accurately than that of the second.

When discussing COBRA-techniques for which the abundance change factor is hardly predictable, it was already said that to know the abundance change factor in advance is convenient, but not necessary. What is important is that abundance change factors remain the same in different experiments, which is meant by the term “reproducible”. In fact the abundance change factor for each selected nucleic acid component can be reproduced, either by the researcher or by someone else working independently (in distinct experimental trials) according to the same reproducible experimental description and procedure. The exact values of abundance change factors may be measured in a control experiment.

Abundance change factors are required to convert concentrations of components in the subsequent nucleic acid mixture, namely the COBRA-library, into the concentrations of correspondent components in the analyzed, original mixture. It is worth noting that for some tasks it is enough to know only concentrations of components in the COBRA-library (so, it is possible to go without abundance change factors). For example:
if the task of biodiversity study is to compare relative representation of some organisms in a series of test samples;
if the purpose of the analysis is to find varying components in a series of test samples for further investigation;
if in pre-developed assay all conclusions (e.g. clinical decisions or biodiversity characteristics) are bound to the concentrations of the components in the COBRA-library and not to the concentrations in the analyzed mixture.

Besides, we already mentioned, that in most cases researchers are interested not in absolute, but in relative abundances.

Useless Sequencing Reads

Over the past two decades, the tendency is that instead of analyzing individual components of nucleic acid mixtures (Northern, RT-PCR, digital PCR) massive analysis of all or substantially all components of mixtures is performed, for example for expression profiling, analysis of biodiversity, etc. Currently such massive analysis is most often performed using microarrays or high-throughput sequencing machines.

The inventors have noticed that microarrays or high-throughput sequencers react differently on the change of composition of analyzed nucleic acid mixture. If some components would be removed from the analyzed mixture it would practically not affect analysis of other components on a microarray. In contrast, in massively parallel sequencing analysis after removal of some component other components would get more sequencing reads. Similarly, if some additional component would be added to the analyzed mixture it would not affect a microarray assay, but would hurt massively parallel sequencing, because this component would “occupy” some of the sequencing reads.

So, unlike to microarrays, efficiency of massively parallel sequencing may be improved by excluding useless components from the analyzed mixture of nucleic acids. Useless components are those, (i) which are completely uninteresting for the researcher, or (ii) which are overrepresented in the mixture. In the first case it would be desirable to remove components from the mixture completely; in the second case it would be desirable to decrease their abundances. We found out, that controllable change of abundance may be accomplished by relatively simple molecular biology procedures.

Despite the fact that the controllable change of abundance may be accomplished by relatively simple molecular biology procedures, this method has never been used to analyze the concentrations of components of nucleic acid mixtures. The generally accepted strategy was either to preserve the composition of the mixture as accurate as possible, or to remove some components completely. A good example is ribosomal RNA. Although ribosomal RNA makes up most of the cellular RNA, analysis of rRNA concentrations is almost never carried out. Instead it is discarded from the analysis. At the same time it is known that rRNA content is not constant and might be important in some biological processes or serve as a diagnostic marker. rRNA would remain in the analysis, if its abundance is reproducibly reduced to some acceptable level. For example, using the inventive methods it is possible to reduce the rRNA concentration. According to the present invention it is preferred to change the relative abundance of the component in a controllable manner instead of eliminating it completely from the analyzed nucleic acid mixture.

Useless reads also occur when sequencers are used for other biomedical applications. Sequencing machines of the previous generation (sequencing by Sanger) were used for the construction of EST-libraries. To catch rare transcripts it was necessary to repeatedly sequence clones corresponding to abundant transcripts (useless reads). To solve the problem, it was proposed to use normalized libraries. In a normalized DNA library all DNAs are represented at comparable frequencies. During their preparation no information about concentration of a single molecule in the original mixture is conserved. There are several protocols for preparation of normalized libraries based on the dependence of the rehybridization rate of nucleic acids on concentration. Attempts have been made to use normalized libraries for comparison of expression profiles. However, this approach is not widely used because of a lot of drawbacks:
the normalization effect is limited: after normalization highly expressed genes still produce more sequencing reads than low expressed ones;
rehybridization rate depends not only on the concentration of the component, but also on its nucleotide sequence;
highly expressed homologues may suppress a low expressed similar transcript because of cross-hybridization;
normalization rate has limited reproducibility, no predictability and strongly depends on the experimental protocol.

Useless reads may appear when sequencing machines are used for sequencing or resequencing of genomic DNA.

Each region in a genome should be read a certain number of times (sequencing coverage). Insufficient coverage is unacceptable because it would lead to inaccurate results. If for a particular genomic region excessive (relative to a required coverage) reads are generated, they will be useless.

Useless reads may occur due to the errors in sequencing planning, if the total number of reads is too large for the size of a particular genome.

In some cases sequencing of the entire genomic DNA would inevitably result in a too large portion of useless reads. For example, in clinical studies it is required to know the nucleotide sequences not of the entire genome but of certain areas of the genome. Special methods are developed to prepare sequencing libraries containing only particular regions of the genome, e.g. multiplex PCR, hybridization-based enrichment.

Another source of useless reads is a distortion of uniform representation of components of mixture which should be sequenced. Distortion may be a result of non-uniform amplification or of non-uniform hybridization-based selection. Before distortion, all genomic regions have same abundances. After distortion, some regions become more abundant than others. To reach the required sequencing coverage for rare components, the abundant ones should be over-sequenced. Usually, researchers put efforts to prevent such distortion, for example:
using linear amplification methods (in vitro transcription, RCA, etc.),
using limited rates of exponential amplification (PCR, BRCA, etc.),
designing multicomponent PCR in such a way that amplification of different components is as equal as possible;
performing hybridization-based selection long enough to achieve saturation.

Although in the discussed methods regarding sequencing and resequencing of genomic DNA (as in the current invention) the idea is to avoid useless sequencing reads, they differ from the methods of the present invention. “Sequencing/resequencing of genomic DNA” on one side and “analyzing concentrations of the components of nucleic acid mixture” are different research tasks. Besides, the main idea in “sequencing/resequencing” is to preserve the abundance of analyzed components: either of the entire genome or of the regions required to be sequenced.

Thus the methods according to the present invention suitable for expression profiling preferably refer to nucleic acids in the original mixture selected from the group comprising or consisting of: RNA, total RNA, mRNA, mtRNA, rRNA, tRNA, dsRNA, small RNA/micro RNA, and cDNA.

If the method according to the present invention is used for analysis of biodiversity, it is preferred that the nucleic acid of the original mixture is selected from the group comprising or consisting of: RNA or DNA from an environmental or clinical sample.

Different next generation sequencing platforms are used in biomedicine. Effectiveness of all of them may be improved by decreasing the amount of useless sequencing reads. Besides, there are other detection technologies, which are sensitive to the presence of useless components in the analyzed mixture, for example the long known serial analysis of gene expression or recently appeared digital color-coded barcode technology. Efficiency of all methods of concentration measurement which are sensitive to the presence of useless components in the analyzed mixture may be improved by using COBRA-approach.

### DESCRIPTION OF THE FIGURES

FIG. 1: Scheme of the COBRA approach. A. Traditional sequencing library. The abundance of cDNA fragments matches the abundance of transcript in the analyzed mixture. B. COBRA approach. The abundances of molecules in COBRA sequencing library are adjusted according to the required accuracy of concentration measurement. Suppression levels for each component are shown on the graph. Concentrations of components in the analyzed mixture may be determined by multiplying concentrations in the COBRA-library on corresponding suppression levels.

FIG. 2: Different number of detectable loci for different components of nucleic acid mixture. Contour arrows show components of nucleic acid mixture “α” and “β” which have different concentration. Solid arrows correspond to locus-specific oligonucleotides used for preparation of sequencing library. A. In case there is one detector locus per component the number of sequencing reads corresponding to components “α” and “β” considerably differs. B. If more detector loci are selected for the rare component, the number of sequencing reads corresponding to components “α” and “β” is comparable.

FIG. 3: Stepwise decrease of dynamic range of concentrations. Components of the nucleic acid mixture with dynamic range of concentrations of five orders of magnitude are assigned to three groups according to their level of abundance (shown in black, grey and white). COBRA-sequencing libraries are prepared using three different library preparation protocols “without suppression”, “10× suppression” and “100× suppression”. Dynamic range of concentrations of COBRA library molecules is three orders of magnitude.

FIG. 4: Abundance adjustment using a mixture of functional and blocked locus-specific oligonucleotides. Locus “α”: all oligonucleotides are functional, no suppression occurs. Locus “β”: a mixture of functional and blocked oligonucleotides (1:4). Because of competition for the template, the yield of library molecules will decrease in 5 times.

FIG. 5: Schemes of cDNA synthesis methods using functional and blocked locus-specific oligonucleotides in primer extension reaction. A. 80% blocking of the first strand synthesis. Locus-specific oligonucleotides (functional/blocked=1:4) are used for cDNA synthesis. B. 80% blocking of the second strand synthesis. Locus-specific oligonucleotides (functional/blocked=1:4) are used for initiation of second strand synthesis. In both cases only fifth part of transcripts results in corresponding ds cDNA molecules.

FIG. 6: Schemes of methods using functional and blocked locus-specific oligonucleotides. A. Gap-filling. B. Allele-specific ligation C. DANSR.

FIG. 7: Using of biotinylated primers as blocked primers. Sequencing library molecules are prepared using a mixture of biotinylated and non-biotinylated (4:1) locus-specific oligonucleotides. As a result the fifth part of library molecules is not biotinylated. Before sequencing, biotinylated molecules are removed, and non-biotinylated are sequenced, providing a 80% suppression.

FIG. 8: Using of dUTP-containing primers as blocked primers. Sequencing library is prepared using sets of locus-specific oligonucleotides, three per locus. They need to be ligated to produce a library molecule. To regulate the representation of library molecules corresponding to a certain locus, a mixture of “functional” internal oligonucleotides (with standard nucleotides) and “blocked” oligonucleotides (containing uridines in the T positions) is used. Both types of internal oligonucleotide participate in ligation, however library molecules with “blocked” oligonucleotide are destroyed by UDGase prior to sequencing. The ratio of standard oligonucleotide to the uridine-containing one determines the level of suppression.

FIG. 9: Using primers with conservative 5′ region as functional locus-specific oligonucleotides. Sequencing library is prepared using sets of locus-specific oligonucleotides, three per locus. They need to be ligated and then amplified to produce a library molecule. To regulate the representation of library molecules corresponding to a certain locus, a mixture of functional upstream oligonucleotides (with conservative 5′ region) and blocked oligonucleotides (without conservative 5′ region) is used. Both types of upstream oligonucleotide participate in ligation, however library molecules with blocked oligonucleotide don't have a binding region for the PCR primer and can't be amplified. The ratio of oligonucleotides with and without the 5′ tail for amplification determines the level of suppression.

FIG. 10: The use of different abundance regulation schemes under different conditions. A. Limited amount of starting material. B. The amount of starting material is sufficient to obtain reliable data for the “rare” loci.

FIG. 11: Scheme of digital analysis of selected regions (DANSR). For each locus of interest a set of three locus-specific oligonucleotides is used. They need to be ligated to produce a molecule with 5′ and 3′ regions correspondent to sequencing adapters.

FIG. 12: Ligation of detector oligonucleotides on RNA template. For each locus of interest a set of three locus-specific oligonucleotides is used. They need to be ligated to produce a molecule with 5′ and 3′ regions correspondent to sequencing adapters. Reverse transcription is done before the amplification.

FIG. 13: Suppression of individual loci due to performing reaction with part of the original material. A. Separation of original material before primers addition. B. Reaction scheme avoiding unwanted suppression of rare loci.

FIG. 14: Different number of cycles in cyclic ligation reaction for different adjustment level groups of loci. A. Standard scheme of cyclic ligation. All detector oligonucleotides are added in the beginning of cyclic ligation. B. COBRA cyclic ligation. Detector oligonucleotides corresponding to different adjustment level groups are introduced into a cyclic ligase reaction after different numbers of cycles.

FIG. 15: Different number of PCR cycles for different adjustment level groups of loci. A. Structure of ligated DANSR detector oligonucleotides for COBRA PCR amplification. Regions of flanked detector oligonucleotides correspondent to adjustment level groups are used for group-specific PCR. B. COBRA PCR amplification. PCR primers corresponding to different adjustment level groups are introduced into amplification reaction after different numbers of cycles.

FIG. 16: Stepwise positive COBRA selection. There are three groups of selector oligonucleotides: (i) “rare” group without adjustment level correction; (ii) “intermediate” group with 10× abundance suppression; (iii) “abundant” group with 100× abundance suppression. Analyzed NA mixture is divided into portions correspondent to the abundance suppression level. “Rare” group is added to the whole NA mixture. “Intermediate” group is added to the 10% of the NA mixture. “Abundant” group is added to the 1% of the NA mixture. Selector oligonucleotides bind to the correspondent library molecules. Selected molecules are combined together to prepare a COBRA-library.

FIG. 17: Scheme of DANSR methods using functional and blocked primers. A. To regulate the representation of library molecules corresponding to a certain locus, a mixture of functional and blocked internal oligonucleotides is used. If blocked oligonucleotide is annealed to the template, ligation would not occur. B. Structure of internal DANSR primers. 3′ and 5′ ends of “blocked” primer are modified to prevent ligation.

FIG. 18: Positive COBRA selection with functional/blocked primers. Abundance adjustment level may be individually selected for each locus. Functional selector oligonucleotides are biotinylated. Blocked selector oligonucleotides are not biotinylated.

FIG. 19: Stepwise negative COBRA selection. There are two groups of selector oligonucleotides: (i) “intermediate” group with 10× abundance suppression; (ii) “abundant” group with 100× abundance suppression. In contrast to FIG. 16 there is no “rare” selector oligonucleotide group: all untargeted transcripts remain for the analysis. Analyzed NA mixture is divided into portions correspondent to the abundance suppression level. “Intermediate” group is added to the 90% of the NA mixture. “Abundant” group is added to the 99% of the NA mixture. Selector oligonucleotides bind to the correspondent RNA molecules. Selected molecules are removed from the analyzed mixture. The result of the negative selection is a COBRA RNA mixture. Any sequencing procedure may be used for the analysis of this COBRA RNA mixture.

FIG. 20: Negative COBRA selection with functional/blocked primers. Abundance adjustment level may be individually selected for each locus. “Functional” selector oligonucleotides are not biotinylated. “Blocked” selector oligonucleotides are biotinylated.

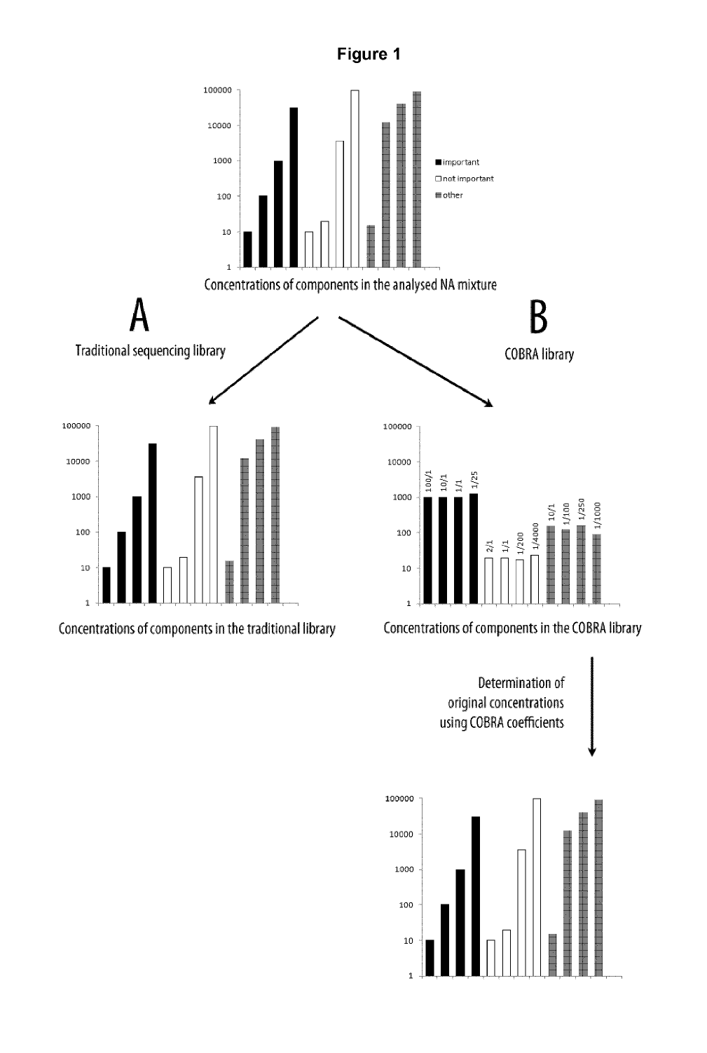

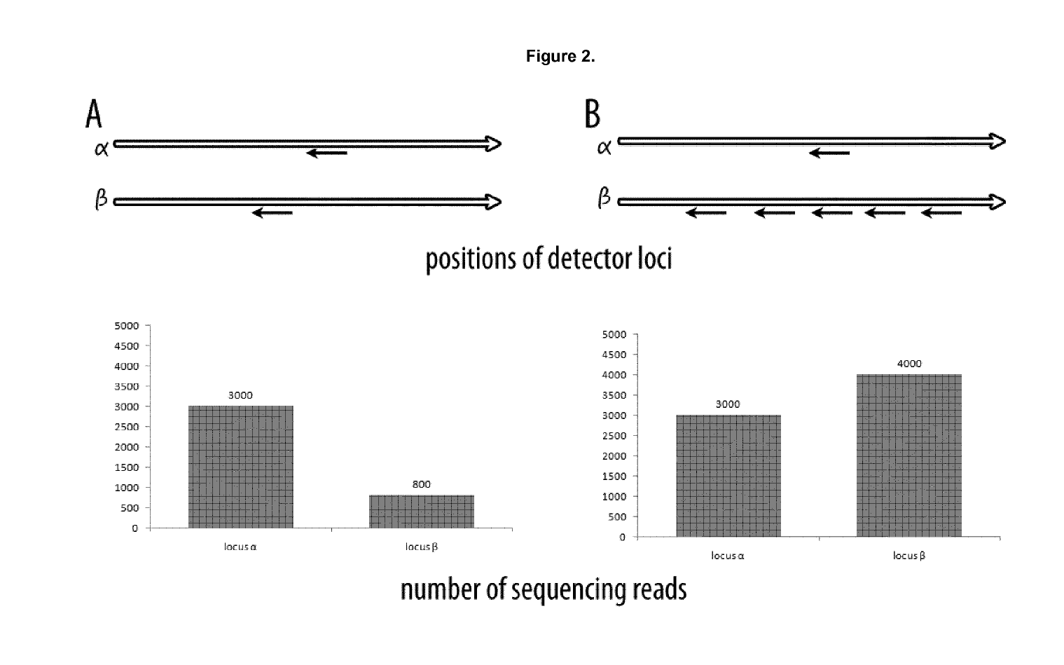

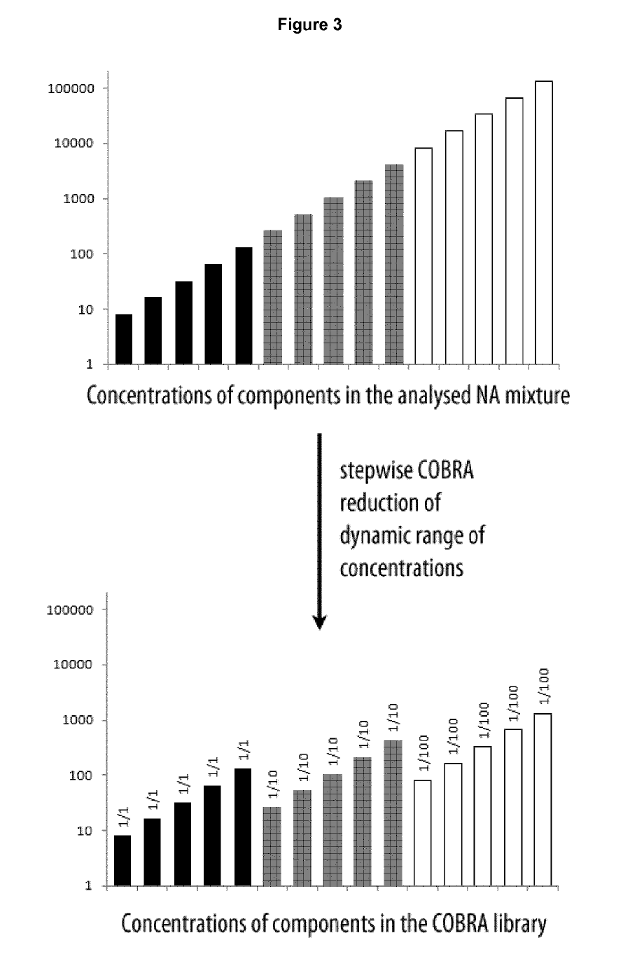

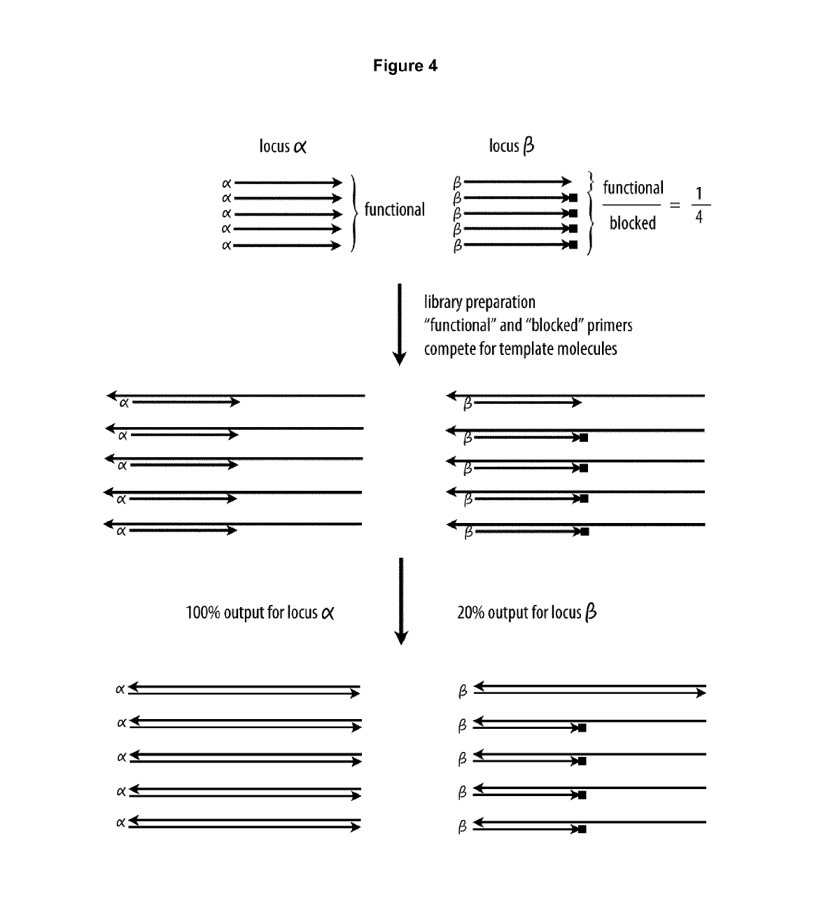

### EXAMPLES

### Example 1

### Preparation of the Sequencing Library by Ligation of Detector Oligonucleotides on a cDNA Template

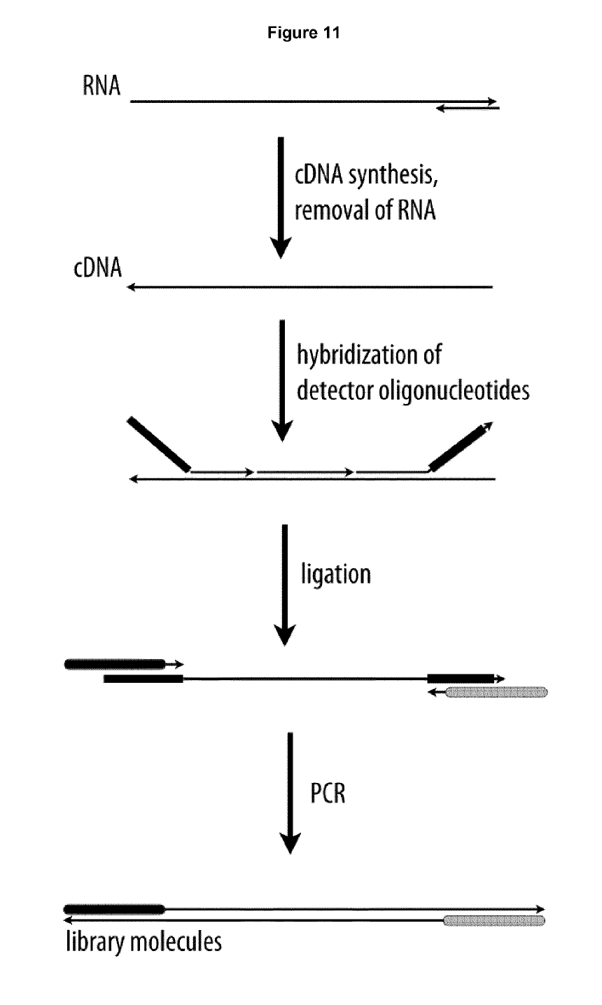

The scheme of the sequencing library preparation is shown in FIG. 11. After cDNA synthesis and RNA removal, selected loci are detected by cDNA-dependent ligation of locus-specific detector oligonucleotides. Three detector oligonucleotides are used for each locus. Flanking oligonucleotides contain regions corresponding to the sequencing library adapters: 5′-region of the upstream oligonucleotide and 3′-region of the downstream oligonucleotide.

Following ligation and getting rid of most of the non-ligated oligonucleotides the library amplification is performed. During amplification ligated molecules acquire full-size sequencing adapters.

Sequencing is used for detection, accounting and quality control of library molecules. If a sequenced molecule contains fragments belonging to different loci or fragments are ligated in the wrong order, it is excluded from the further analysis.

### Example 2

### Preparation of the Sequencing Library by Ligation of Detector Oligonucleotides on a RNA Template

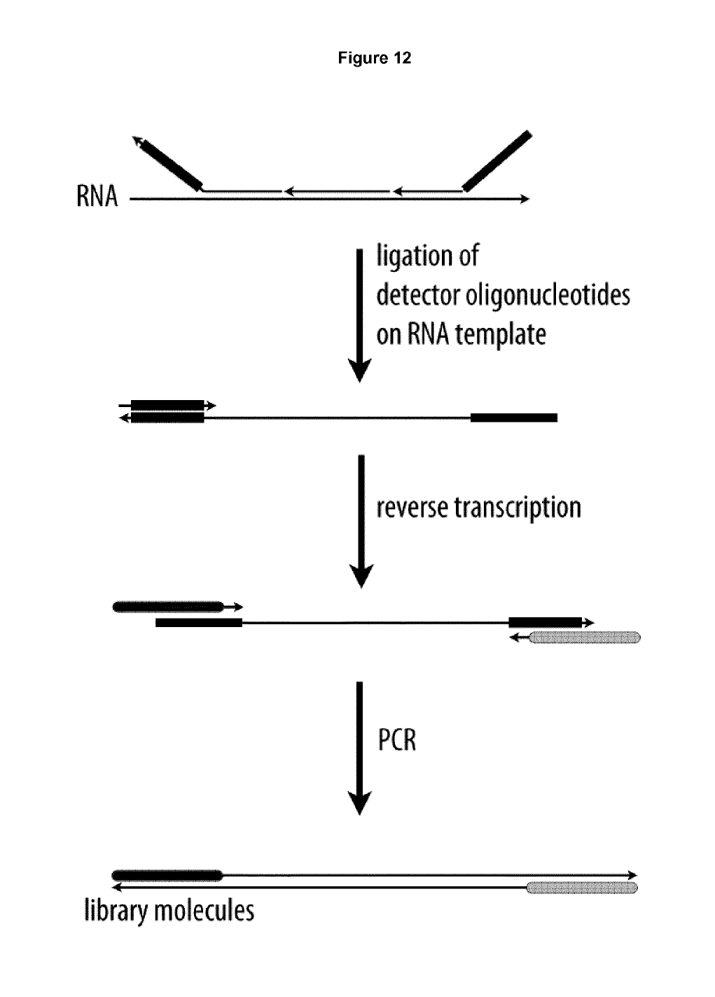

T4Rnl2 RNA ligase enzyme can be used for ligation of detector oligonucleotides directly on the RNA template [2]. FIG. 12 shows the scheme of the corresponding protocol. For efficient ligation it is necessary that at least 3′-regions of upstream and middle oligonucleotides consist of ribonucleotides. Library molecules are obtained after reverse transcription of the ligated oligonucleotides.

Following ligation, getting rid of most of the non-ligated oligonucleotides and reverse transcription the library amplification is performed. During amplification ligated molecules acquire full-size sequencing adapters.

Sequencing is used for detection, accounting and quality control of library molecules. If a sequenced molecule contains fragments belonging to different loci or fragments are ligated in the wrong order, it is excluded from the further analysis.

### Example 3

### Separate Reactions for Different Groups of Detector Oligonucleotides

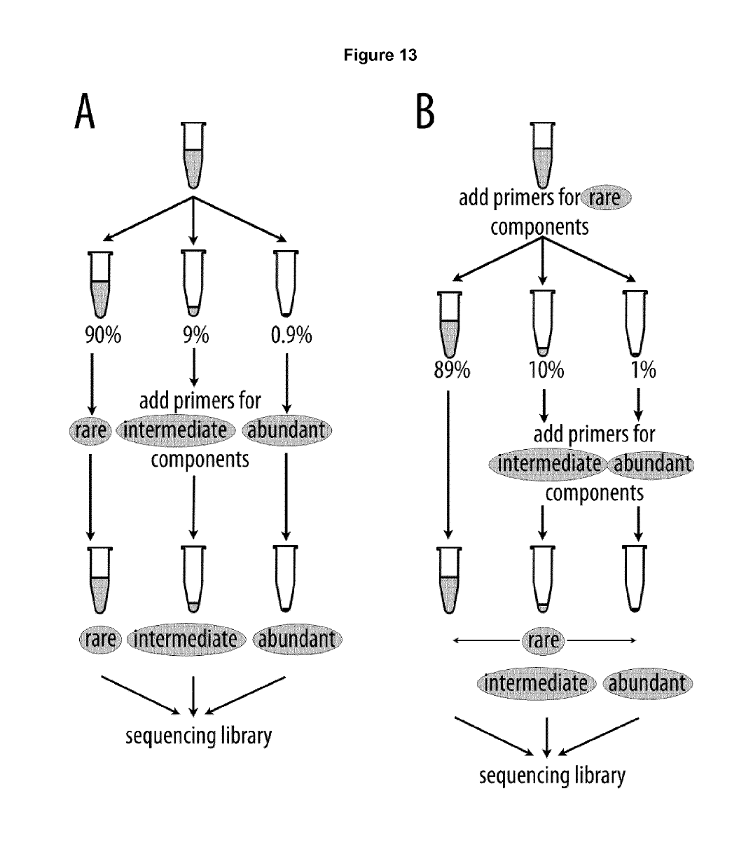

Genes with “high”, “intermediate” and “low” levels of expression were selected, 10 genes in each group. Using the procedure described in Example 1 two sequencing libraries were prepared. When preparing the first library primers for all loci were used together. For the preparation of the second library reaction mixture was divided into three separate reactions, as shown in FIG. 13A.

It was found that the frequency of sequencing reads corresponding to genes with a “high” and “intermediate” levels of expression is reduced in the second library 100 and 10 times respectively.

### Example 4

### Separate Reactions for Different Groups of Detector Oligonucleotides

COBRA library was prepared using the same primers as in the Example 3, but the reaction mixture was divided, as shown in FIG. 13B.

In Example 3 some unwanted suppression occurs, since ˜10% of the starting material is inaccessible to the primers corresponding to low expressed genes. On the scheme shown in FIG. 13B, suppressed are only those genes that really need to be suppressed.

### Example 5

### Different Number of Ligation Cycles for Different Groups of Primers

When using a thermostable ligase (e.g. Pfu or Taq ligase) detection reaction described in the Example 1 can be performed cyclically, each cycle consisting of steps of denaturation, annealing and ligation. This allows to obtain several library molecules from each template cDNA.

It is possible to change the relative abundance of the library molecules corresponding to different adjustment level groups, if corresponding groups of locus-specific detector oligonucleotides are introduced into a cyclic ligase reaction after different numbers of cycles. The earlier detector oligonucleotides are introduced into the cyclic ligase reaction, the more library molecules would be obtained from each template cDNA.

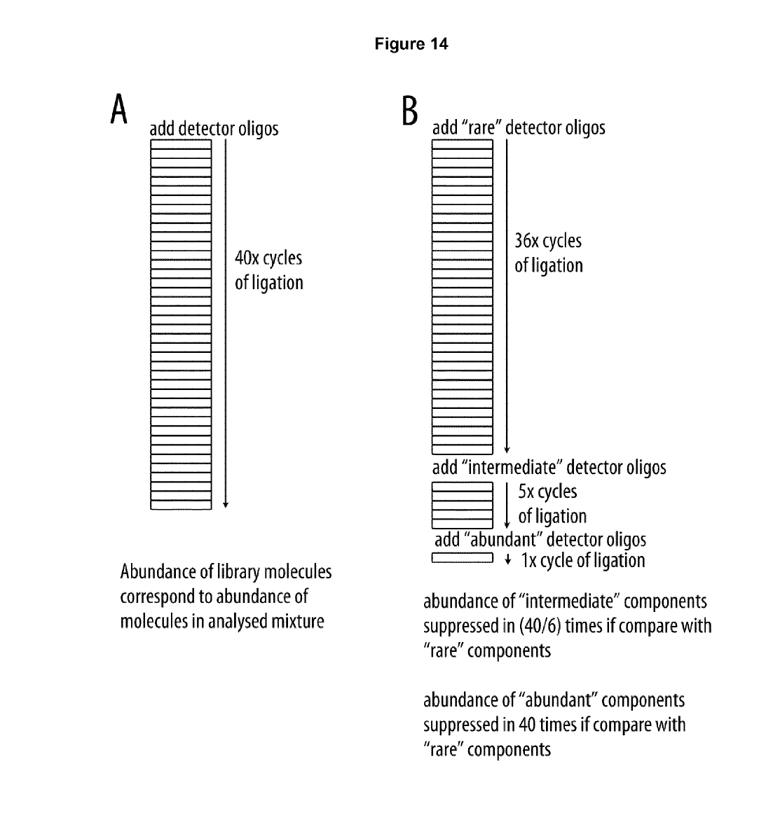

On the scheme shown in FIG. 14 relative concentrations of abundant and intermediate groups of loci fell 40 and 6.7 times respectively due to the different number of ligation cycles for different groups of primers:
40 for primers corresponding to rare loci;
6 for primers corresponding to intermediate loci;
1 for primers corresponding to abundant loci.

### Example 6

### Different Number of Amplification Cycles for Different Groups of Primers

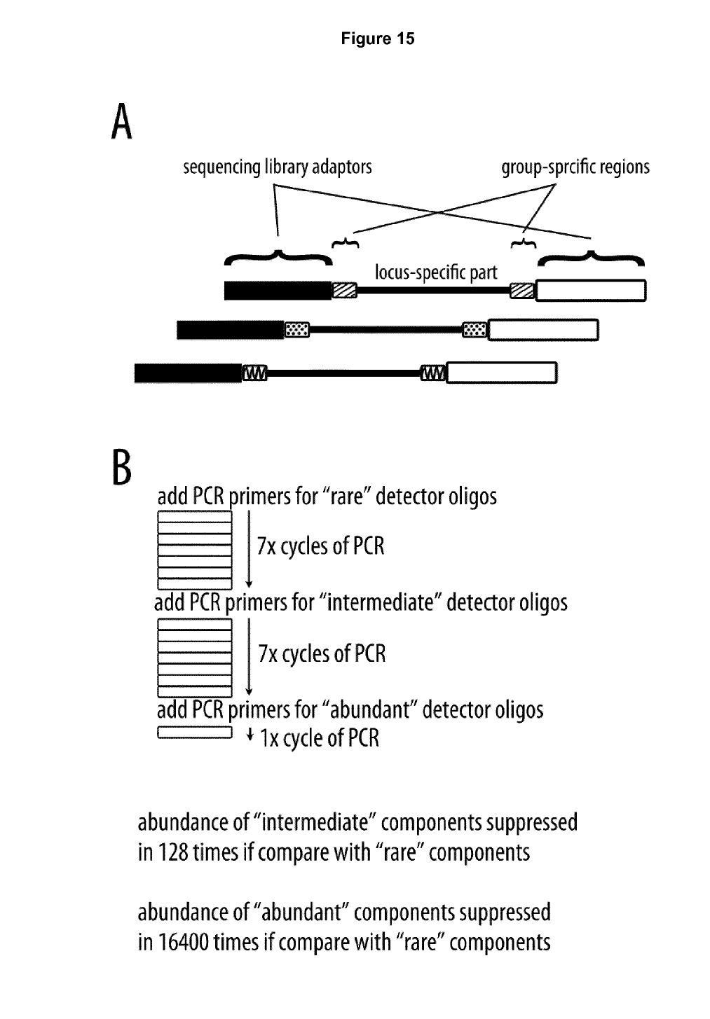

If oligonucleotides used in the reaction described in Example 1 have the structure shown in FIG. 15A, a stepwise change of relative concentrations of different groups of transcripts can be carried out at the stage of library preamplification. To provide group-specific amplification 3′ ends of group-specific PCR-primers should correspond to group-specific regions of ligated detector oligonucleotides.

As in the previous example, group-specific PCR-primers should be added on different PCR cycles (FIG. 15B). Marker region would provide selective amplification of specific library molecules.

On the scheme shown in FIG. 15 relative concentrations of abundant and intermediate groups of loci fell 16400 and 128 times respectively due to the different number of cycles of PCR for different groups of primers:
15 for primers corresponding to rare loci;
8 for primers corresponding to intermediate loci;
1 for primers corresponding to abundant loci.

Examples 5 and 6 show how stepwise level adjustment can be carried out in a common reaction mixture. In Example 5, spatial isolation of oligonucleotides from different adjustment level groups is used, and in Example 6 oligonucleotides of different adjustment level groups have different markers.

### Example 7

### Stepwise COBRA Selection of RNA-Seq Library Molecules

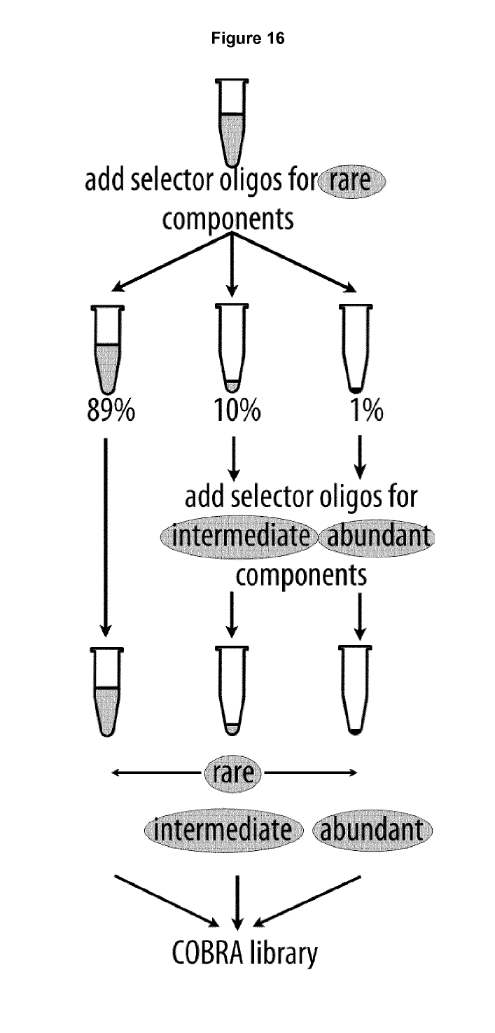

COBRA changing of abundance can be carried out directly prior to sequencing of a standard RNA-Seq library. The scheme is shown in FIG. 16. Hybridization with biotinylated locus-specific selector oligonucleotides is performed to fish out transcripts of interest from RNA-Seq library.

Library is divided into portions. For each portion selector primers belonging to groups with corresponding adjustment levels are applied. Relative abundance of different transcripts is changed because only a certain part of the library is available to selector oligonucleotides from a particular adjustment level group.

Performing COBRA-procedure prior to sequencing is convenient because:
the procedure can be easily adapted to different protocols of RNA-Seq library preparation;
only one selector oligonucleotide per locus is required;
when selector oligonucleotide is long enough, procedure is not sensitive to point mutations located in the hybridizing region;
for standard applications standard sets of selector oligonucleotides can be used.

For example, in the routine clinical analysis only a few types of human tissues are easily available (blood, saliva, buccal cells, sperm, feces). For each of these tissues, appropriate COBRA selector oligonucleotides can be designed.

### Example 8

### Functional/Blocked Primers: Arrested Primer Extension

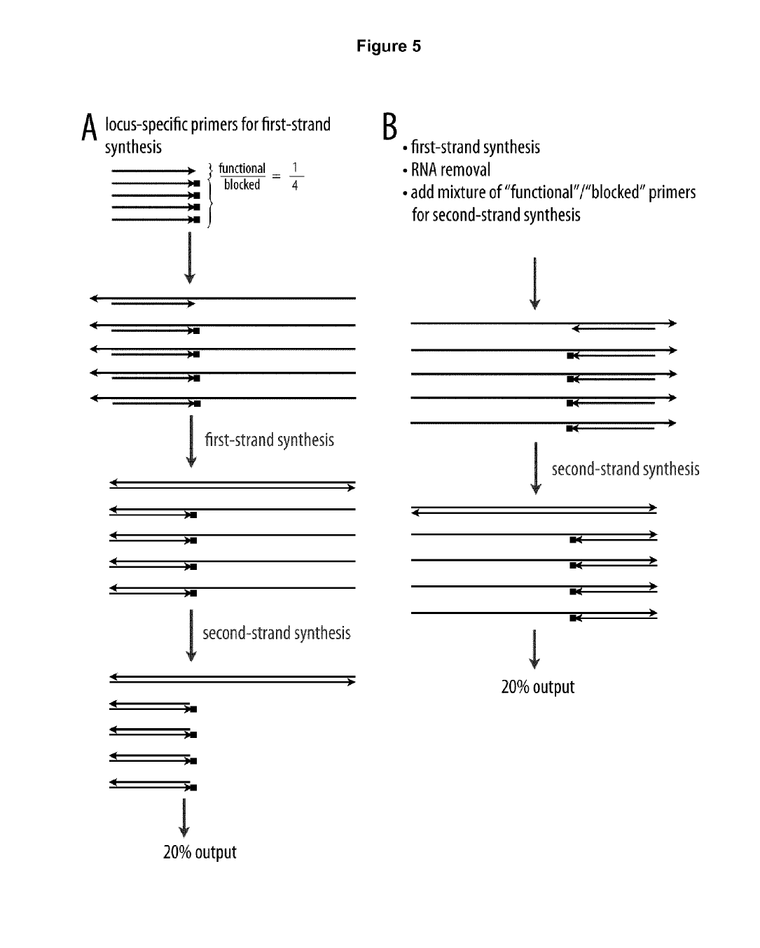

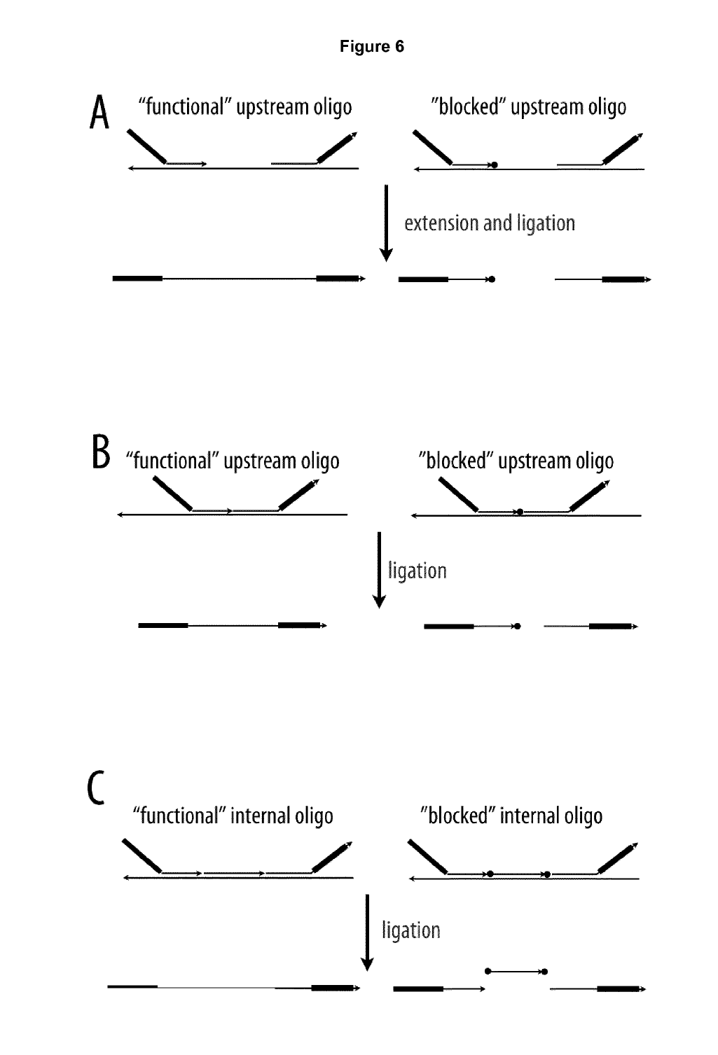

Examples of using blocked primers which are unable to participate in primer extension reaction are shown in FIGS. 5A, 5B and 6A.

FIG. 5A shows the protocol for preparation of a COBRA RNA-Seq library with a partial blocking of the first strand synthesis. Among the advantages of the method is the small number of primers (one per locus), which however can cause high background. Obtained library molecules are heterogeneous, which can be inconvenient for the analysis of the sequencing data.

To reduce the number of molecules of the library, synthesized from non-specific primers, it makes sense to use primers with the 5′ part correspondent to the sequencing adapter. Then during the preparation of the library, only the second sequencing adapter should be ligated.

FIG. 5B shows a protocol with blocked synthesis of the second strand. If 5′ parts of the primers used for first- and second-strand synthesis are conservative and correspond to sequencing adapters, library molecules are obtained immediately after synthesis of the second strand.

FIG. 6A shows a scheme of gap-filling reaction. This approach is useful to analyze polymorphic regions. If “blocked” primer can't be extended in the course of primer extension reaction, a gap between the detector oligonucleotides would remain, and ligation would not occur.

### Example 9

### Functional/Blocked Primers: Arrested Primer Ligation

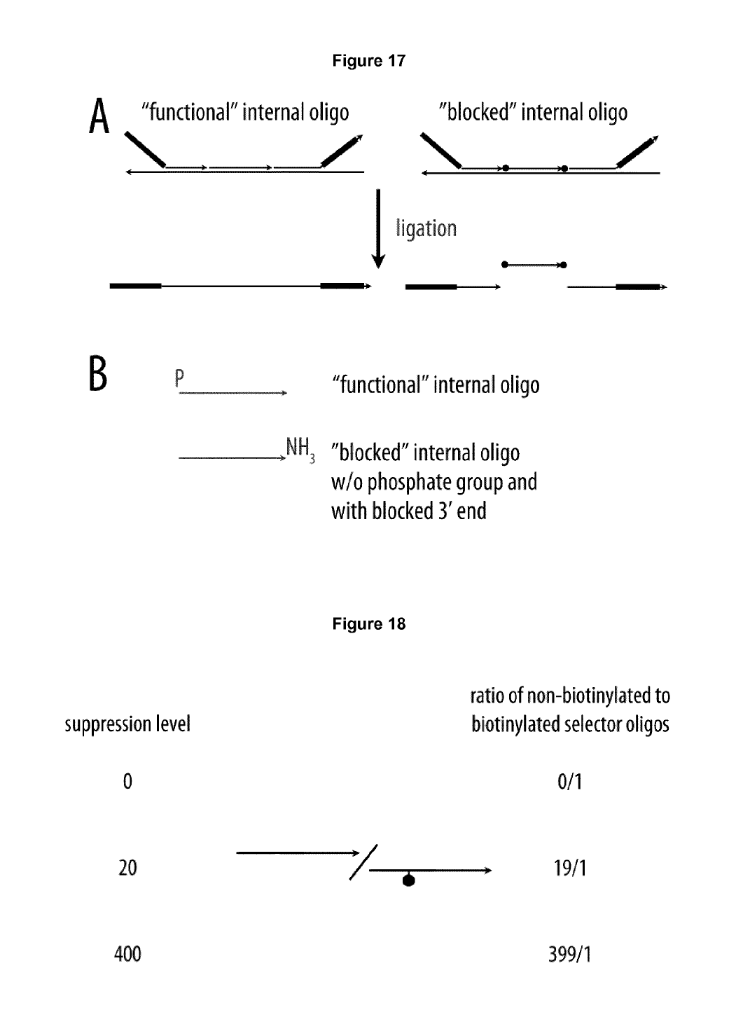

Examples of using blocked primers which are unable to participate in ligation reaction are shown in FIGS. 6B, 6C and 17.

The use of two (or three) specific primers for each locus reduces the number of non-specific molecules in the library. If it is necessary to analyze a polymorphic region, the ligation can be combined with a gap-filling reaction (FIG. 6A).

If 5′- parts of upstream and 3′-parts of downstream primers are conservative and correspond to sequencing adapters, library molecules are obtained immediately after ligation.

COBRA library was made according to the protocol described in Example 1 using a mixture of functional/blocked primers (FIG. 17). The structure of blocked primers is shown in FIG. 17B. For primers corresponding to genes with “high”, “intermediate” and “low” levels of expression the ratio of functional/blocked primers was “1:99”, “1:9” and “1:0”, respectively.

It was found out that the frequency of sequencing reads corresponding to genes with “high” and “intermediate” level of expression is reduced in 100 and 10 times respectively.

### Example 10

### Selectable Blocked and Functional Library Molecules

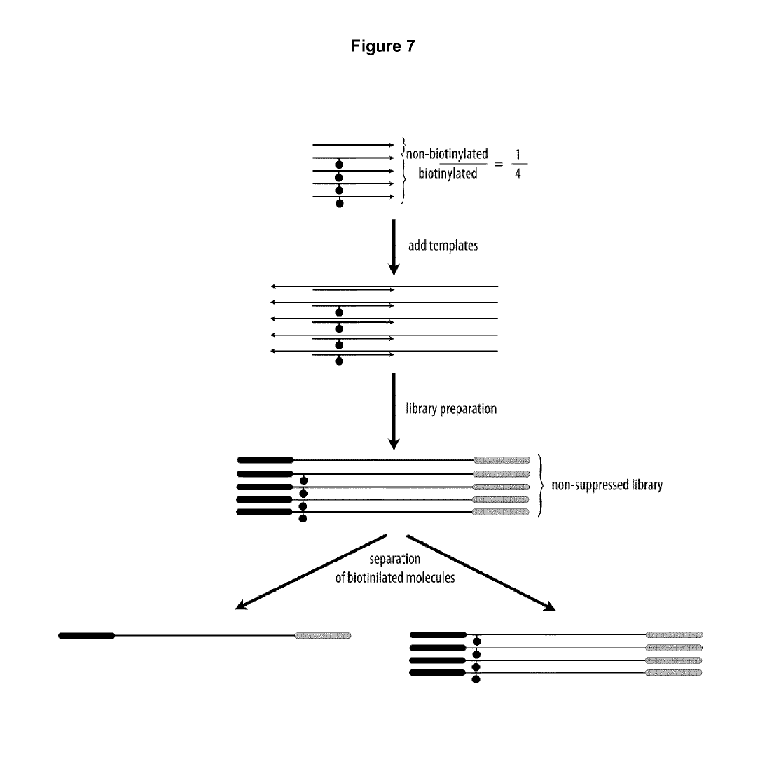

Schemes of methods that allow to separate the molecules produced in the reaction with the participation of functional primers from the molecules derived from reactions with blocked primers are shown in FIGS. 7, 8, and 9.

FIG. 7 shows a protocol where “blocked” primers are biotinylated—corresponding library molecules can be bound to streptavidin coated particles and excluded from sequencing.

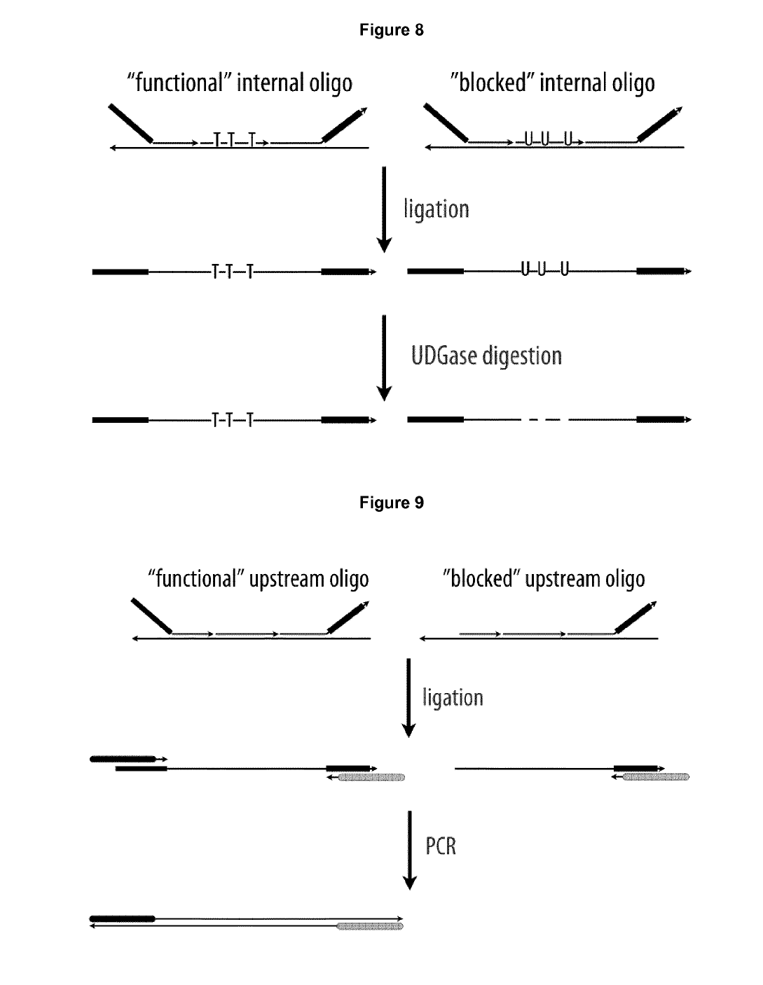

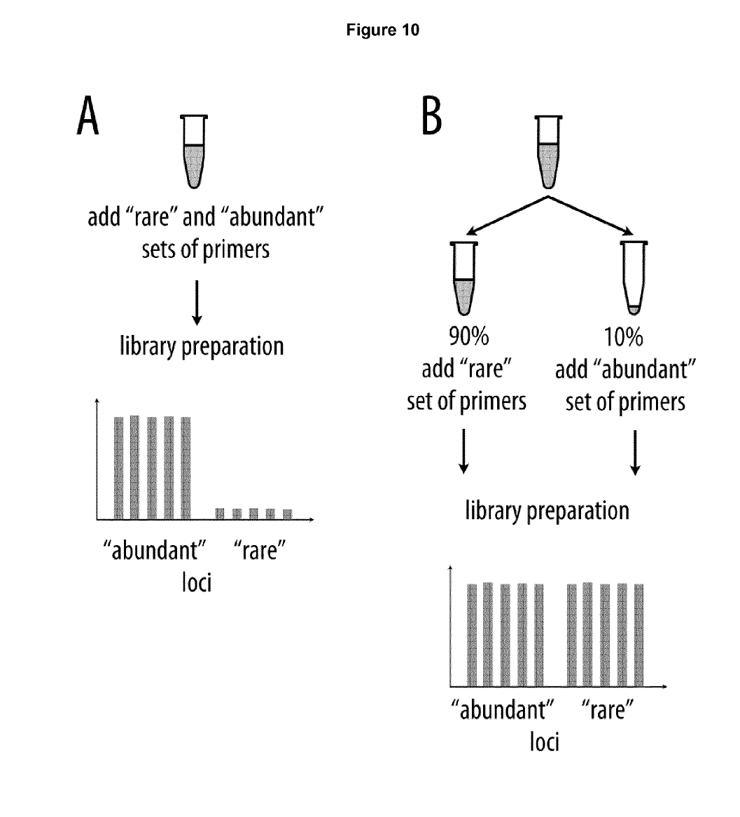

FIG. 8 shows a protocol where blocked primers contain uridine—corresponding library molecules can be destroyed by UDGase. Library molecules originating from the “functional” primers withstand UDGase treatment.

FIG. 9 shows the protocol where functional upstream detector oligonucleotides contain a conservative 5′ region (for further amplification of library molecules). Blocked upstream detector oligonucleotides do not contain such a region. After ligation, amplification is carried out using primers corresponding to conservative regions. Library molecules originating from the functional primers are amplified, and, besides acquire full-size sequencing adapters.

### Example 11

### Functional/Blocked Primers: COBRA Selection of RNA-Seq Library Molecules

The use of functional/blocked oligonucleotides allows to perform COBRA-selection of RNA-Seq library molecules before sequencing without splitting the reaction mixture into portions (as in Example 7). Sets of “functional” and “blocked” selector oligonucleotides for different suppression levels are shown in FIG. 18. “Functional” selector oligonucleotides are biotinylated and library molecules hybridized to them can be fished out and sequenced. Blocked selector oligonucleotides are not biotinylated: they do not allow to fish out library molecules and to prevent binding of library molecules to biotinylated selector oligonucleotides. Proportion of molecules selected for sequencing is determined individually for each locus by the ratio of concentrations of locus-specific biotinylated and non-biotinylated oligonucleotides.

### Example 12

### Functional/Blocked Primers: Negative COBRA-Selection of RNA Molecules for Preparation of Sequencing Library

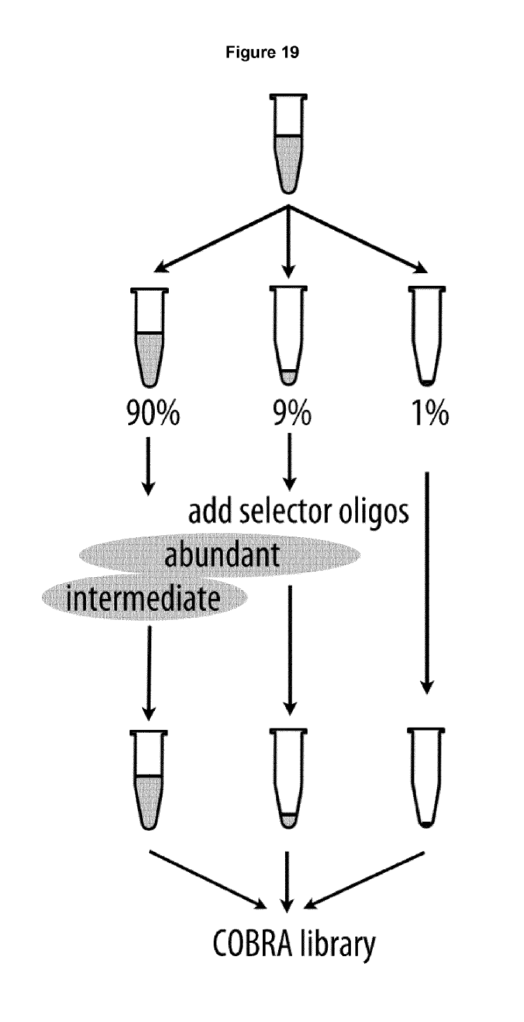

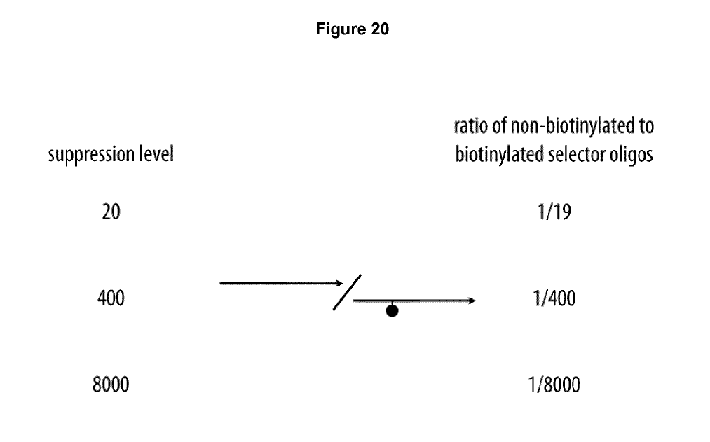

FIGS. 19 and 20 show the implementation of hypothesis-free COBRA procedures using stepwise abundance adjustment (FIG. 19) and using “functional/blocked” selector oligonucleotides for abundance adjustment individually for each locus (FIG. 20). Hypothesis-free COBRA approach is based on the removal of a certain part of transcripts, which otherwise get over-sequenced.

For stepwise level adjustment original mixture is divided into portions and selector oligonucleotides corresponding to different adjustment levels are added to the portions, as shown in FIG. 19. Since for each adjustment level group some part of the mixture remains inaccessible to selector oligonucleotides, a certain portion of transcripts remains for the analysis.

Negative selection can be performed in a single tube without division into portions, if functional/blocked selector oligonucleotides are used (FIG. 20). Performing selection in one tube reduces handwork and makes comparison of the concentrations of various transcripts more reliable. Functional selector oligonucleotides are not biotinylated, they prevent hybridization of the transcripts with biotinylated selector oligonucleotides. Which portion of transcripts remains in the mix for later analysis is determined individually for each locus by the concentration ratio of locus-specific biotinylated and non-biotinylated oligonucleotides.

### REFERENCES

1. Bullard D R, Bowater R P. Direct comparison of nick-joining activity of the nucleic acid ligases from bacteriophage T4. Biochem J. 2006 Aug. 15; 398(1):135-44.

2. Sparks A B, Wang E T, Struble C A, Barrett W, Stokowski R, McBride C, Zahn J, Lee K, Shen N, Doshi J, Sun M, Garrison J, Sandler J, Hollemon D, Pattee P, Tomita-Mitchell A, Mitchell M, Stuelpnagel J, Song K, Oliphant A. Selective analysis of cell-free DNA in maternal blood for evaluation of fetal trisomy. Prenat Diagn. 2012 January; 32(1):3-9. doi: 10.1002/pd. 2922. Epub 2012 Jan. 6.

3. US 2009/1246760A1 (Harris Timothy et al.)

1. A method for analysis of concentrations of components of nucleic acid mixtures by sequencing, wherein relative abundances of at least two components for which concentrations should be measured is changed before sequencing in a reproducible way using locus-specific oligonucleotides and wherein said change of abundances comprises:
i) selection of at least two nucleic acid components of the original mixture for which concentrations should be measured and relative abundances should be changed and designing locus-specific oligonucleotides for said at least two nucleic acid components;
ii) creation from original nucleic acid mixture a subsequent nucleic acid mixture wherein relative abundances of components corresponding to the components selected in i) are changed in a reproducible manner using said locus-specific oligonucleotides.

2. The method according to claim 1, wherein relative abundances of components corresponding to the components selected in i) are changed in ii) by
a) using differing number of locus-specific oligonucleotide sets for said components, and/or
b) using for these components differing reaction conditions, and/or
c) using for these components mixtures of functional and blocked locus-specific oligonucleotides with differing ratio of said “functional to blocked” locus-specific oligonucleotides, and/or
d) by using for these components locus-specific oligonucleotides with differing concentrations or with differing efficiency of hybridization.

3. The method according to claim 2, wherein said differences in reaction conditions of b) are selected from different amounts of original mixture containing nucleic acids used in reactions; different number of cycles in cyclic amplification reactions; and different reaction times in linear amplification reactions.

4. The method according to claim 2, wherein the functional oligonucleotides of c) can be elongated in reaction of primer extension, or reaction of first-strand synthesis, or reaction of second-strand synthesis, or in PCR, or in gap-filling reaction because they have 3′ end modification, and the blocked oligonucleotides of c) cannot be elongated in reaction of primer extension, or reaction of first-strand synthesis, or reaction of second-strand synthesis, or in PCR, or in gap-filling reaction because they have 3′ end modification.

5. The method according to claim 2, wherein the functional oligonucleotides of c) can participate in ligation steps of ligation detection reaction, or in gap-filling reaction, or in LCR, or in DANSR because they have 3′ or 5′ end modifications, and the blocked oligonucleotides of c) cannot participate in ligation steps of ligation detection reaction, or in gap-filling reaction, or in LCR, or in DANSR because they have 3′ or 5′ end modifications.

6. The method according to claim 2, wherein the functional oligonucleotides and/or the blocked oligonucleotides have markers selected from:
presence in oligonucleotide of dUTP for subsequent specific destruction;
presence in oligonucleotide of thio-modified bonds for subsequent specific destruction;
presence in oligonucleotide of biotin for subsequent specific affinity selection;
presence in oligonucleotide of 5-bromo-2′-deoxyuridine for subsequent specific affinity selection; and
presence in oligonucleotides of sequence specific for subsequent amplification or hybridization-based selection.

7. The method according to claim 1, wherein the relative abundances of components selected in i) are changed in such a way, that the dynamic range of concentrations of components under analysis in the subsequent nucleic acid mixture is lower than the dynamic range of concentrations of components under analysis in the original mixture containing nucleic acids, wherein the relative abundances of components selected in i) are changed in such a way which decreases the abundance of components for which concentration without change of abundances is measured with excessive accuracy and/or increases the abundance of components for which it is desirable to increase the accuracy of concentration measurement if compared with measurement of concentration without change of abundances.

8. The method according to claim 1, wherein the subsequent nucleic acid mixture is selected from: sequencing library, set of ligated locus-specific oligonucleotides, set of locus-specific oligonucleotides extended in a template-dependent reaction, set of fluorescently labeled molecules, and nucleic acids molecules selected with the help of hybridization with locus-specific oligonucleotides.

9. The method according to claim 1, wherein the relative concentrations of components under analysis in the original nucleic acid mixture are calculated by dividing results obtained after changing of abundances by correspondent abundant change factors.

10. The method according to claim 1, wherein the subsequent nucleic acid mixture is created by positive selection with locus-specific oligonucleotides and contains only components corresponding to locus-specific oligonucleotides while all other nucleic acid components of original mixture are removed, or wherein subsequent nucleic acid mixture is created by negative selection with locus-specific oligonucleotides and in the subsequent nucleic acid mixture relative abundances are changed only for components corresponding to locus-specific oligonucleotides.

11. The method according to claim 1, wherein the nucleic acid of the original mixture is selected from the group consisting of: RNA, total RNA, mRNA, mtRNA, rRNA, tRNA, dsRNA, small RNA/micro RNA, and cDNA.

12. The method according to claim 1, wherein the nucleic acid of the original mixture is selected from the group consisting of: RNA or DNA from an environmental or clinical sample.

13. A method for analyses of biodiversity or expression profiling in medicine, veterinary, agriculture, or ecological studies comprising the method according to claim 1.

14. A kit comprising a functional and blocked locus-specific oligonucleotide sets, wherein the functional and blocked locus-specific oligonucleotide sets are used in the method according to claim 1.

15. The method of claim 11, wherein the method is utilized for expression profiling.

16. The method of claim 12, wherein the method is utilized for analysis of biodiversity.
